(19) United States
(12) Patent Application Publication
(10) Pub. No.: US 2022/0389439 A1
(43) Pub. Date: Dec. 8, 2022

(54) TRANSGENIC PLANTS FROM THE BRASSICA spp. GENUS WITH MYCORRHIZATION CAPACITY AND HAVING AN INCREASED PRODUCTIVITY

(71) Applicant: UNIVERSIDAD DE SALAMANCA, Salamanca (ES)

(72) Inventors: Carlos NICOLÁS RODRÍGUEZ, Salamanca (ES); Enrique MONTE VÁZQUEZ, Salamanca (ES); Rosa HERMOSA PRIETO, Salamanca (ES); Ana ALONSO RAMÍREZ, Salamanca (ES); Jorge POVEDA ARIAS, Salamanca (ES)

(21) Appl. No.: 17/776,466

(22) PCT Filed: Nov. 12, 2019
(86) PCT No.: PCT/EP2019/081029

§ 371 Date: May 12, 2022

(51) Int. Cl. C12N 15/82

(52) U.S. Cl. CPC C12N 15/8261; C12N 15/8273; C12N 15/8247

(57) ABSTRACT

The present invention relates to a transgenic plant, preferably of the Brassica spp. genus, comprising in its genome a fungal sequence, preferably belonging to the fungus Trichoderma harzianum, capable of establishing symbiosis with mycorrhizal fungi. Additionally, the transgenic plants of the invention have an increased biomass and higher resistance to abiotic stress. The present invention also provides methods for increasing the resistance to abiotic stress of plants of the Brassica spp. genus, as well as methods for obtaining said transgenic plants with the capacity to establish mycorrhization processes and resistance to abiotic stress and methods for producing foods, feeds or industrial products using the transgenic plant.

The present invention is comprised in the technical field of plant biotechnology, and namely relates to a genetically modified plant which acquires the capacity to establish symbiosis with mycorrhizal fungi, and has a higher increase in its biomass and yield, in addition to higher resistance to abiotic stress, than non-genetically modified, wild-type control plants. Additionally, the invention relates to a method of obtaining genetically modified plants, as well as to methods for producing foods, feeds and/or industrial products using the transgenic plant.

### BACKGROUND OF THE INVENTION

Mycorrhizae are a type of symbiotic association between fungi and vascular plant roots which can be transformed into excellent allies for crops of any type, since they foment their cost-effectiveness because they favour saving resources; allow reducing the water intended for irrigation and fertilisation; promote the biological activity of the soil; and increase crop output; facilitate protection against abiotic and biotic stresses, with a potential application in the biocontrol of phytopathogens and in phytoremediation, among other advantages. Arbuscular mycorrhizal forming fungi (AMF) colonise the intraradical tissue of the host plant, where they develop structures characteristic of symbiosis (arbuscules and vesicles), and extraradical mycelium, which interacts with the ecosystem of the rhizosphere and is responsible for the absorption of nutrients from the soil.

Although most vascular plants are capable of forming symbiosis with mycorrhiza-producing fungi, specifically endomycorrhizae, 18% of them are incapable. Thus, there are two groups of plants not susceptible to mycorrhization, depending on their adaptations to each type of soil. The first of these groups are the Brassicaceae group, including the families Amaranthaceae, Brassicaceae, Caryophyllaceae, Chenopodiaceae, Polygonaceae and Urticaceae, characterised by settling in disturbed soils, where competition with other plants is low and the availability of phosphorus (P) in soil is high, therefore, they lack a specialised root system to access the available P. The nutritional abundance of these soils has induced a selective force towards elimination of mycorrhization, as it is not necessary. The other group is the Proteaceae (Chilean hazelnut and macadamia nut) group, which includes the families Cyperaceae, Haemodoraceae, Proteaceae and Restionaceae, which can live in soils where the availability of P is very low, for the assimilation of which they develop a range of root specialisation, such as cluster roots, dauciform roots, or sand-binding roots, which do not need to symbiotically associate with mycorrhizal fungi. These two groups represent an ecologically significant division, since they are found at both ends of the soil fertility spectrum as far as phosphorus is concerned.

Focusing on the particular case of the Brassicaceae family, it should be pointed out that different studies have been performed to determine why this symbiosis was lost, and it has been concluded that the presence of mycelium in the soils in which these species are developed represented a competitive drawback, since it reduces the root branching and development, in addition to depleting phosphorus levels; however, it is also important to point out that in soils rich in phosphorus, the formation of mycorrhizae is suppressed. It is also known that the absence of mycorrhization in plants of the Brassicaceae family by mycorrhizal fungi does not occur due to the production of glucosinolates by said plants as a strategy of protection against the attack of pathogenic fungi and bacteria. The toxic action of glucosinolates is the result of the myrosinase activity converting them to isothiocyanates. The production of isothiocyanates is a primary strategy of defence.

Among the plants of the Brassicaceae family, rapeseed (Brassica napus), cauliflower (Brassica oleracea var. Botrytis) or mustard (Brassica nigra) crops and model plant Arabidopsis thaliana stand out. Particularly, rapeseed is an oleaginous species which has a high percentage of excellent-quality oil and an extraction residue with a high protein level. At present, it is the most widely cultivated oleaginous plant in the European Union. Worldwide, the largest producers are Canada, China and India. The rapeseed crop takes on considerable importance due to the increase in demand by the biodiesel industry and the price thereof. Rapeseed oil is a raw material of interest for this industry, which has caused the increase in the sown area of this oleaginous plant. Furthermore, rapeseed is used for obtaining oil for human consumption, flour and fodder. Its incorporation in production systems has a number of advantages for the producer and for the industry.

Given the importance of crops from the Brassicaceae family, there is a need in the state of the art to improve their production, seeking for said production to be increasingly greater or for it to require fewer resources to maintain it. Another aspect to be improved to induce a higher yield of these crops is obtaining plants showing higher resistance to abiotic stresses, as well as a production of oil with a suitable composition, specifically having a lower concentration of toxic glucosinolates.

### DESCRIPTION OF THE INVENTION

The present invention relates to the use of a fungal nucleotide sequence coming from the fungus Trichoderma harzianum, to induce in plants, in which it does not occur naturally, the capacity to establish symbiosis with fungi (mycorrhization). Specifically, the present invention relates to the use of the nucleotide sequence comprising SEQ ID NO: 1 and coming from the fungus T. harzianum, with Gen Bank database accession number EU399786.1 for the induction of mycorrhization, preferably in plants of the Brassica spp. genus and more preferably in plants which are selected from the list consisting of: B. napus, B. oleracea, B. nigra and B. rapa, more preferably the plant of the species B. napus.

SEQ ID NO: 1 is the nucleotide sequence of the gene ThKel1 from the fungus T. harzianum encoding the protein ThKEL1 comprising SEQ ID NO: 2, with UniProtKB database accession number: D3T185. Said protein, ThKEL1, has kelch domains, widely distributed in proteins of viral, bacterial and eukaryotic origin. In the context of the present invention, ThKel1 is also defined by a nucleotide or polynucleotide sequence which encodes the amino acid sequence SEQ ID NO: 2, and which additionally would comprise several variants from: a) nucleic acid molecules encoding a polypeptide comprising the amino acid sequence of SEQ ID NO: 2, b) nucleic acid molecules whose complementary hybrid chain hybridises with the polynucleotide sequence of a), c) nucleic acid molecules whose sequence differs from a) and/or b) due to the degeneracy of the genetic code, d) nucleic acid molecules encoding a polypeptide comprising the amino acid sequence with an identity of at least 80, 90, 95, 96, 97, 98 or 99% with SEQ ID NO: 2, and in which the polypeptide encoded by said nucleic acids has the activity and structural characteristics of the ThKEL1 protein. Among said nucleic acid molecules is the sequence SEQ ID NO: 1.

As shown herein, the use of SEQ ID NO: 1 for obtaining genetically modified (transgenic) plants with the capacity to establish symbiosis in the presence of fungi (mycorrhization) (FIGS. 1 and 2), induces an increase in the yield of said transgenic plants (FIG. 6), showing a significant increase in seed production (FIG. 4), which furthermore show a higher weight (FIG. 5) and higher production of siliques per branch (FIG. 3), compared to the seed produced by the corresponding wild-type control plants. Additionally, the transgenic plants of the present invention are more resistant to different types of abiotic stress, such as, for example, saline stress (FIG. 7) or drought (FIGS. 8 to 12).

Another advantages of the use of SEQ ID NO: 1 for obtaining transgenic plants with the aforementioned technical features and capabilities is that said plants present a significant reduction in the amount of glucosinolates they synthesize (FIG. 13), and in their hydrolysis products (FIG. 14), as well as a lower presence of aliphatic glucosinolates in the oil obtained from the seeds of the transgenic plants of the invention (FIG. 15), and a higher content in compounds of nutritional interest (FIG. 16) compared to the corresponding wild-type control plants.

Thus, a first aspect of the present invention relates to the use of SEQ ID NO: 1 in the induction of a mycorrhization process, preferably in plants of the family Brassicaceae, more specifically in plants of the Brassica spp. genus, more preferably in plants which are selected from the list consisting of: B. napus, B. oleracea, B. nigra and B. rapa, and even more preferably in plants of the species B. napus.

Another aspect of the present invention relates to the use of SEQ ID NO: 1 in the production of plants genetically modified (transgenic plants).

As it is used herein, the term “nucleic acid molecule” or “nucleic acid sequence” refers to deoxyribonucleotides or ribonucleotides and polymers thereof in single- or double-stranded form. The terms “nucleic acid molecule” or “nucleic acid sequence” can also be used indistinctly with gene, cDNA and mRNA encoded by a gene. The term “gene” is widely used to refer to any segment of DNA associated with a biological function. Therefore, the genes include encoding sequences and/or the regulating sequences required for their expression. The genes also include unexpressed DNA segments which, for example, form recognition sequences for other proteins. The genes can be obtained from a variety of sources, including cloning from a source of interest or synthesis from known or predicted sequence information, and may include sequences designed to have the desired parameters.

Another aspect of the present invention relates to transgenic plants (hereinafter transgenic plant of the invention), or to the reproductive or propagating material for a transgenic plant (hereinafter reproductive or propagating material of the invention), or to a cultured transgenic plant cell (hereinafter transgenic plant cell of the invention), comprising in its genome a nucleotide sequence comprising SEQ ID NO: 1.

As it is used herein, the term “plant” includes whole plants, any reproductive or propagating material for a plant, progeny of the plants and parts of plants, including seeds, siliques, fruits, leaves, flowers, shoots, stems, roots, isolated plant cells, tissues and organs. References to a plant may also include plant cells, plant protoplasts, plant tissue cultures, plant calluses, plant clusters and plant cells which are intact in plants or parts of plants, such as embryos, pollen, ovules, seeds, leaves, flowers, branches, fruits, grains, spikes, ears, hulls, stems, roots, root tips and the like. The offspring, variants and mutants of any of the transgenic plants described herein are within the scope of the present invention. The seeds of any of said transgenic plants are also included.

As it is used herein, the term “plant cell” includes plant cells derived and/or isolated from plant cell tissue or from plant cell culture. As it is used herein, the term “parts of a plant” includes any part or parts of a plant including the seeds, siliques, fruits, leaves, flowers, shoots, stems and/or roots.

The terms “transformed”, “transgenic” and “recombinant” refer to a host organism such as a plant cell, plant or part of a plant in which an exogenous nucleic acid molecule has been introduced. The nucleic acid molecule can be integrated in a stable manner into the genome of the host or the nucleic acid molecule can be present as an extrachromosomal molecule. It is understood that transformed plant cells, plants or parts of plants cover not only the final product of a transformation process, but also the transgenic progeny thereof.

A host “no transformed”, “no transgenic” or “no recombinant” refers to a wild-type organism, for example, a plant cell, plant or part of a plant that does not contain the exogenous nucleic acid molecule.

The term “exogenous” when used herein refers to a nucleic acid molecule (for example, a DNA sequence), gene or protein, which originates from a particular source that is foreign to the cell, plant or part of a plant in which it is introduced. The exogenous nucleic acid molecule can be introduced in the plant in a stable or transient manner to produce an RNA molecule and/or a polypeptide molecule. An “endogenous” nucleic acid molecule, gene or protein of is a nucleic acid molecule (for example, a DNA sequence), gene or protein associated in a natural manner with, or native of, a plant cell, plant or part of a plant.

For purposes of the present invention, the term “genetically modified plant” (transgenic plant) refers to plants the genetic material of which has been deliberately modified so that they can express the protein encoded by the gene ThKel1. Specifically, the transgenic plants of the invention have been modified by genetic engineering to have an increased expression of the gene ThKel1 described herein. For purposes of the present invention, the plant cell of a transgenic plant comprises the polynucleotide of the present invention. The term “transgenic plant” includes whole plants, parts of plants (stems, roots, leaves, fruits, etc.) or organs, plant cells, seeds and progeny thereof. A transformed plant can be a direct transfectant, which means that the DNA construct was introduced directly in the plant, such as through Agrobacterium or other methods, or the plant can be the progeny of a transfected plant. The later or second-generation plants can be produced by sexual reproduction, i.e. fertilisation. Furthermore, the plants can be a gametophyte (haploid stage) or a sporophytes (diploid stage).

The term “corresponding non-genetically modified plant” (corresponding wild-type plant, or corresponding wild-type control plant) refers in the present invention to plants for which the genetic material has not been modified to have an increased expression of the gene ThKel1 described herein. Said definition applies likewise to a control cell. In one embodiment, an example of a control plant or control host cell is one that is wild-type. In one embodiment, an example of a control plant or control host cell is one that is not of the wild-type (for example, it is transgenic for any other type of coding region) but has not been designed to have an increased expression of the protein encoded by the gene ThKel1, described herein.

For purposes of the present invention, the term “reproductive or propagating material” refers to any material capable of giving rise to a complete plant or to parts thereof. Said materials are obtained by methods of vegetative reproduction (for example, layers of air or dirt, division, grafting, micropropagation, stolons or runners, storage organs such as bulbs, corms, tubercles and rhizomes, hitting or cutting, twinning), sexual reproduction (breeding with another plant) and asexual reproduction (e.g. apomixis, somatic hybridisation).

For purposes of the present invention, the transgenic plants, the reproductive or propagating material, or the plant cell, genetically modified, as described herein, preferably belong to the family Brassicaceae, more preferably, genus Brassica spp, even more preferably to the species which are selected from the list consisting of: B. napus, B. oleracea, B. rapa and B. nigra, and even more preferably, the species B. napus.

In a preferred embodiment, the transgenic plants, the reproductive or propagating material, or the plant cell of the present invention may comprise in its genome more than one copy of SEQ ID NO: 1.

In another preferred embodiment, the transgenic plants, the reproductive or propagating material, or the plant cell, have the capacity to establish mycorrhization processes, and a higher tolerance to abiotic stress compared to the corresponding wild-type control plants.

For purposes of the present invention, the term abiotic stress refers to the damage caused by salinity, drought and nutritional deficiencies.

In another preferred embodiment, the transgenic plants, the reproductive or propagating material, or the plant cell, of the invention, show an increase in their biomass and yield compared to the corresponding wild-type control plants.

As it is used herein, the term “biomass”, “biomass of a plant” or “plant biomass” refers to the amount of a tissue produced by a plant. An increase in the biomass of the plant can be in the complete plant or in parts thereof, such as aerial (harvestable) parts, seeds, siliques, fruits, leaves, flowers, stems and/or roots. The biomass of the plant can be measured, for example, by fresh weight and/or dry weight.

As it is used herein, the term “increasing the biomass” or “increase in biomass” means that the plant or parts thereof have increased in size, height and/or mass compared to the corresponding wild-type plant (i.e., a plant not transformed with the exogenous nucleic acid described herein) or compared with a predetermined standard.

As it is used herein, the term “increase in seed production” refers to the increase in the number of seeds per plant, the number of siliques per plant, the number of flowers per plant and/or the number of seeds per silique compared to the corresponding wild-type or control plant, or compared to a predetermined standard. The term also refers to increasing the size of the seed and/or the length of the seed compared to with the corresponding wild-type or control plant, or compared to a predetermined standard.

In another preferred embodiment, the transgenic plants, the reproductive or propagating material, or the plant cell, of the invention, show an increase in the production of oil of their seeds compared to the seed from the corresponding wild-type control plants.

As it is used herein, the term “increase in the production of oil” refers to increasing the oil content of a plant, the oil content of the seeds of a plant and/or the oil content per seed compared to the corresponding wild-type control plant or compared to a predetermined standard.

As it is used herein, the term “increment”, “increase” or “improve” refers to at least an increase of 2, 5, 10, 20, 25, 30, 35, 40, 45, 50, 55, 60, 65, 70, 75, 80, 85, 90, 95 and 100% in biomass, production of seeds and/or production of oil compared to the corresponding wild-type control plant or to a predetermined standard.

In another preferred embodiment of the invention, the transgenic plants, the reproductive or propagating material, or the plant cell, of the invention, show a decrease in the synthesis of glucosinolates, preferably aliphatic and indole glucosinolates, compared to the glucosinolates produced by the corresponding wild-type control plant as well as the levels of toxic isothiocyanates derived from the hydrolysis of glucosinolates.

As it is used herein, the term “reduce” or “decrease” refers to at least at drop of 2, 5, 10, 20, 25, 30, 35, 40, 45, 50, 55, 60, 65, 70, 75, 80, 85, 90, 95 and 100% in the concentration of glucosinolates of a transgenic plant of the invention compared to the corresponding wild-type control plant or to a predetermined standard.

Glucosinolates are sulfur- and nitrogen-containing glycosides which are present in some plants in a natural manner as a result of the secondary metabolism thereof. They are innocuous compounds, but when the plant is attacked, enzymes such as myrosinase come into contact with them, generating glucose, sulfuric acid and toxic compounds such as isothiocyanates, epithio nitriles, simple nitriles or thiocyanates. The defensive function of the glucosinolate-myrosinase system has primarily been attributed to the toxicity of isothiocyanates.

Another aspect of the present invention as described hereinabove relates to a cell, silique, seed, progeny or part of a plant preferably selected from a leaf, a stem, a flower, an ovary, a fruit or a callus, obtained from the transgenic plant of the present invention and characterised in that it comprises SEQ ID NO: 1.

Another aspect of the present invention relates to a method for increasing the biomass and yield, seed production and/or production of oil of a wild-type plant preferably belonging to the family Brassicaceae, more preferably to the genus Brassica, and more preferably to the species which are selected from the list consisting of: B. napus, B. oleracea, B. nigra and B. rapa, even more preferably to the species B. napus, wherein the method comprises:
(a) transforming said wild-type plant with an expression vector comprising the nucleotide sequence SEQ ID NO: 1, and
(b) expressing the transformed nucleic acid molecule in said plant.

As it is used herein, the term “transformation” or “transforming” refers to a process for introducing exogenous nucleic acids in a cell of a plant or part of the plant. Different techniques for introduction polynucleotides in plants are known. In an embodiment of the present invention, the plants are “transformed in a stable manner” with a polynucleotide or DNA construct which comprises it, i.e. the polynucleotide or DNA construct introduced in the plant cell is integrated into the genome of the plant and has hereditary capacity through the offspring thereof. Transformation protocols for introducing polynucleotides or DNA constructs in plant cells may vary depending on the type of plant in question. Suitable transformation methods include, without limitation, microinjection, electroporation, transformation mediated by Agrobacterium and ballistic particle acceleration. Methods for the directed insertion of a polynucleotide or DNA construct at a specific location in the genome of the plant using site-specific recombination systems are also known in the art. Through the application of techniques such as these, the cells of almost any species can be transformed in a stable manner. In the case of multicellular species, transgenic cells can be regenerated in transgenic organisms.

The most widely used method for introducing an expression vector in plants is based on the natural transformation system of Agrobacterium. A. tumefaciens and A. rhizogenes are bacteria in the soil that are pathogenic for plants and genetically transform plant cells.

Numerous transformation vectors available for the transformation of plants are known to those skilled in the art, and the nucleic acid molecules relevant to this description can be used together with any of said vectors. The selection of the vector will depend on the preferred transformation technique and on the species targeted for transformation.

Another aspect of the present invention relates to a method for the production of transgenic plants as described in the present invention, wherein said method comprises:
(a) transforming a wild-type plant with an expression vector comprising nucleotide sequence SEQ ID NO: 1, and
(b) expressing the transformed nucleic acid molecule in said plant.

Alternatively, the present invention also relates to the use of SEQ ID NO: 1 for increasing the biomass and yield, seed production and/or the production of oil of a plant, preferably belonging to the family Brassicaceae, more preferably to the genus Brassica and even more preferably to the species which are selected from the list consisting of: B. napus, B. oleracea, B. nigra and B. rapa, even more preferably to the species B. napus.

Another preferred embodiment relates to the use of the transgenic plants of the invention for the production of foods, feeds or industrial products.

Another aspect of the present invention relates to a method for the production of foods, feeds/and/or industrial products, wherein the method comprises:
(a) obtaining the transgenic plant or a part of same, as described in the present invention, and
(b) preparing the food, feed and/or industrial product from the plant or parts of same.

In a preferred embodiment of the use or method for the production of food, feed and/or industrial product described above, the food or feed is preferably oil, rapeseed oil, semolina, grain, starch, flour or protein. In another preferred embodiment, the industrial product is biofuel, fibre, industrial chemicals, a pharmaceutical product or a nutraceutical.

Another aspect of the present invention relates to an oil production method, preferably rapeseed oil, which comprises:
(a) obtaining the transgenic seeds as described herein,
(b) grinding the seed of step (a) and
(c) extracting the oil from the seed of step (b).

Throughout the description and the claims, the word “comprises” and its variants do not intend to exclude other technical features, additives, components or steps. For those skilled in the art, other objects, advantages and features of the invention may be inferred from both the description and the embodiment of the invention. The following examples and figures are provided by way of example and are not intended to limit the present invention.

### BRIEF DESCRIPTION OF THE FIGURES

FIG. 1 Different rapeseed roots transformed with the gene Thkel1 of T. harzianum subject to mycorrhization with different fungi of the species Glomus spp. (A) G. mosseae; (B) G. microagreatum; (C) G. etunicatum; (D) G. intraradices. The fungus was stained with the ‘Chinese ink’ method. The arrows indicate the vesicles formed by colonisation of the fungus.

FIG. 2 Real-Time PCR quantification of the mycorrhization of rapeseed plants with Glomus spp. Control (wild-type) and transgenic rapeseed plants comprising one copy (KEL1) or two copies (KEL2) of the gene ThKel1 (SEQ ID NO: 1). Quantification of the fungus is represented on the y-axis by means of the ratio between the amount of DNA in μg thereof for every 100 mg of plant tissue.

FIG. 3 Number of siliques per branch in control rapeseed and Thkel1 transgenic rapeseed with mycorrhizae. Both the number of fully developed siliques per branch and those that were aborted and did not form are shown. The bars represent standard deviations of the mean values of all the branches. The asterisk indicates significant differences (p 0.05) between the transgenic lines and the control.

FIG. 4 Number of seeds per silique in control rapeseed plants and in Thkel1 transgenic rapeseed plants with mycorrhizae. The number of seeds per silique is indicated on the y-axis. The bars represent standard deviations of the mean values of all the siliques. The asterisk indicates significant differences (p≤0.05) between the transgenic lines and the control.

FIG. 5 Weight per seed in control rapeseed plants (C) and Thkel1 transgenic rapeseed plants (K) with mycorrhizae (G) or without mycorrhizae. The bars represent standard deviations of the mean values of all the seeds. The asterisk indicates significant differences (p≤0.05) between the transgenic lines and the control.

FIG. 6 Output in control rapeseed plants (C) and Thkel1 transgenic rapeseed plants (K) with mycorrhizae (G) or without mycorrhizae. The bars represent standard deviations. The asterisk indicates significant differences (p≤0.05) between the transgenic lines and the control.

FIG. 7 Siliques per plant and percentage of aborted siliques, under conditions of saline stress. Control rapeseed plants and transgenic rapeseed plants (KEL) (+G, plants treated with fungi Glomus spp.). The bars represent standard deviations. The asterisk indicates significant differences (p≤0.05) between the transgenic lines and the control. KEL1: transgenic rapeseed plants transformed with a copy of the gene ThKel1 (SEQ ID NO: 1). KEL2: transgenic rapeseed plants transformed with two copies of the gene ThKel1 (SEQ ID NO: 1).

FIG. 8 Siliques per plant and percentage of aborted siliques, under conditions of drought stress. Control rapeseed plants and transgenic rapeseed plants (KEL) (+G, plants treated with fungi Glomus spp.). The bars represent standard deviations. The asterisk indicates significant differences (p≤0.05) between the transgenic lines and the control. KEL1: transgenic rapeseed plants transformed with a copy of the gene ThKel1 (SEQ ID NO: 1). KEL2: transgenic rapeseed plants transformed with two copies of the gene ThKel1 (SEQ ID NO: 1).

FIG. 9 Seeds per silique under conditions of drought stress. Control rapeseed plants and transgenic rapeseed plants (KEL) (+G, plants treated with fungi Glomus spp.). The bars represent standard deviations. The asterisk indicates significant differences (p≤0.05) between the transgenic lines and the control. KEL1: transgenic rapeseed plants transformed with a copy of the gene ThKel1 (SEQ ID NO: 1). KEL2: transgenic rapeseed plants transformed with two copies of the gene ThKel1 (SEQ ID NO: 1).

FIG. 10 Seeds per plant under conditions of drought stress. Control rapeseed plants and transgenic rapeseed plants (KEL) (+G, plants treated with fungi Glomus spp.). The bars represent standard deviations. The asterisk indicates significant differences (p≤0.05) between the transgenic lines and the control. KEL1: transgenic rapeseed plants transformed with a copy of the gene ThKel1 (SEQ ID NO: 1). KEL2: transgenic rapeseed plants transformed with two copies of the gene ThKel1 (SEQ ID NO: 1).

FIG. 11 Weight per seed under conditions of drought stress. Control rapeseed plants and transgenic rapeseed plants (KEL) (+G, plants treated with fungi Glomus spp.). The bars represent standard deviations. The asterisk indicates significant differences (p≤0.05) between the transgenic lines and the control. KEL1: transgenic rapeseed plants transformed with a copy of the gene ThKel1 (SEQ ID NO: 1). KEL2: transgenic rapeseed plants transformed with two copies of the gene ThKel1 (SEQ ID NO: 1).

FIG. 12 Output under conditions of drought stress. Control rapeseed plants and transgenic rapeseed plants (KEL) (+G, plants treated with fungi Glomus spp.). The bars represent standard deviations. The asterisk indicates significant differences (p≤0.05) between the transgenic lines and the control. KEL1: transgenic rapeseed plants transformed with a copy of the gene ThKel1 (SEQ ID NO: 1). KEL2: transgenic rapeseed plants transformed with two copies of the gene ThKel1 (SEQ ID NO: 1).

FIG. 13 Determination of glucosinolates (A) 4-methoxyl-I3M (4-methoxyl-indole-3-methyl), (B) 1-methoxy-I3M (1-methoxy-indole-3-methyl) and (C) 4-hydroxy-I3M (4-hydroxy-indole-3-methyl), in wild-type rapeseed plants (C) and transgenic rapeseed plants (Kel2) without mycorrhizae treatment and after three weeks of treatment with mycorrhizae Glomus spp. (+G). The bars represent standard deviations. The asterisk indicates significant differences (p≤0.05) between the transgenic lines and the control. The results show the normalized peak area/mg of tissue.

FIG. 14 Determination of the hydrolysis products of glucosinolates (A) 1-MeO-IGL (1-methoxy-indole-3-methyl) and (B) 4-MeO-IGL (4-methoxyl-indole-3-methyl), in wild-type rapeseed plants (C) and transgenic rapeseed plants (Kel2) without mycorrhizae treatment and after three weeks of treatment with mycorrhizae Glomus spp. (+G). The bars represent standard deviations. The asterisk indicates significant differences (p≤0.05) between the transgenic lines and the control. The results show the normalized peak area/mg of tissue.

FIG. 15 Quantification of the levels of glucosinolates (4-MTB) (4-methylthio-3-butenyl), (B) 5-MTP (5-methylthiopentyl) and (C) 4-MeO-I3M (4-methoxyl-indole-3-methyl) in the oils of rapeseed seeds. C: wild-type rapeseed plants; Kel: transgenic rapeseed plants; +G: wild-type or transgenic rapeseed plants in contact with Glomus spp. The values were grouped together according to significant differences with respect to one another (p<0.05) by means of Tukey's test, in capital letters on the columns.

FIG. 16 Quantification of the levels of the lipids (A) octadecatrienoic acid and (B) glycerophosphocholine in the oils of rapeseed plant seeds. C: wild-type rapeseed plants; Kel: transgenic rapeseed plants; +G: wild-type or transgenic rapeseed plants in contact with Glomus spp. The values were grouped together according to significant differences with respect to one another (P<0.05) by means of Tukey's test, in capital letters on the columns.

### EXAMPLES

The invention is illustrated below by means of tests conducted by the inventors that demonstrate the effectiveness of the product of the invention.

Biological Material Used.

The seeds used belong to the species B. napus spring Jura variety.

For the mycorrhization tests, a formulation based on mycorrhizal fungi Glomus spp. specifically G. mosseae, G. microagreatum, G. etunicatum, G. intraradices and G. claroideum called MIRATEXT-02 (Mirat S. A., Salamanca, Spain), has been used.

### Example 1. Obtaining the Thk1 Transgenic Rapeseed Plants of the Invention

Cloning by Gateway Technology

This technology allows introducing an insert of interest in a vector without having to cut with restriction enzymes and binding. Firstly, the oligonucleotides for amplifying the fragment are designed to be cloned by introducing recombination sites attB1 (GGGGACAAGTTTGTACAAAAAAGCAGGCTGC—SEQ ID NO: 3) at the 5′ end and attB2 (GGGGACCACTTTGTACAAGAAAGCTGGGTC—SEQ ID NO: 4) at the 3′ end. Once PCR has been performed, the obtained fragment is introduced in the pDONOR201 plasmid by the Gateway BP Clonase II Enzyme mix (Invitrogen) (introduces segments of DNA with attB sites in a donor vector with attP sites), by recombination, incubating the mixture overnight at 25° C., now being referred to as pENTR201 plasmid. The reaction is stopped by adding proteinase K (it digests keratin) for 10 min at 37° C.

Next, competent DH5a cells were transformed with the recombination mixture by electroporation. To that end, an aliquot of DH5a cells together with the mixture was placed in a cuvette (0.1 cm wide), and was put in the electroporator (1.8 kV, 1 kW and 25 μF). After that, the bacteria recovered from the cuvette were transferred to LB medium, where they were incubated at 37° C. under stirring a 250 rpm for 1 hour in order to regenerate. After that time, they were seeded on plates with LB medium supplemented with kanamycin, keeping the plates at 37° C. overnight. From the colonies that grew, some colonies were selected and inoculated with LB media supplemented with the same antibiotic, and were kept for 24 h under stirring (250 rpm) and at 37° C. for multiplication.

The plasmid DNA was extracted with the Nucleospin Plasmid kit (Macherery-Nagel), following the supplier's instructions. This method is based on the use of columns designed for extracting purified plasmid DNA. Next, the concentration of the DNA was determined in the Nanodrop and viewed in agarose gel.

The plasmid DNAs were sent to be sequenced using the oligos Seq-Gate.L1.F (TCGCGTTAACGCTAGCATGCATGGATCTC— SEQ ID NO: 5) and Seq-Gate.L2.R (GTAACATCAGAGATTTTGAGACAC— SEQ ID NO: 6) designed for the attL1 and attL2 recombination sites which are generated in the pENTR201 vector and flank the DNA sequence introduced.

Once the sequence of the pENTR201 plasmid has been checked, it was introduced in a DESTINY plasmid (with the right and left border of Agrobacterium tumefaciens). The plasmid used in this process was pKGWFS7. The recombination of the pENTRY and pDEST plasmids was performed with LR clonase (it introduces a gene of interest flanked by attL sites of an ENTRY vector in a destination vector with attR sites), incubating the mixture at 25° C. overnight. The reaction was stopped by adding proteinase K for 10 min at 37° C.

Next, competent DH5a cells were transformed with the recombination mixture by electroporation (1.8 kV), this time seeding the bacteria obtained in LB medium supplemented with spectinomycin, since this is the resistance the destination plasmid presents. After selecting the transformed colonies three different PCRs were performed to confirm the construct (presence of the cloned fragment and said fragment being in the right direction and in the suitable vector), to that end oligos attB1 (SEQ ID NO: 3) and attB2 (SEQ ID NO: 4), Seq-Gate.L1.F (SEQ ID NO: 5) and Seq-Gate.L2.R (SEQ ID NO: 6), oligos of the cloned fragment SEQ ID No: 8 and SEQ ID No: 9) and another oligo of the destination vector (35S-GTW F) were used. The obtained PCR products were checked in agarose gel, a band being observed only in the first and third PCRs.

Lastly, the plasmid DNA was extracted and purified with the NucleoSpin Plasmid (Macherey-Nagel) kit, quantifying the product in the Nanodrop and checking it by agarose gel electrophoresis.

Transformation of A. tumefaciens by Means of Electroporation

A. tumefaciens competent cells were transformed with the pKGWFS7-Thkel1 construct by electroporation. Between 0.1-0.5 μg of the DNA of the recombinant plasmid were added to a suspension of 0.4 ml of competent cells. Immediately after electroporation, 1 ml of SOC medium (2% bacto tryptone, 0.5% yeast extract, 0.05% NaCl, 2.5 mM KCl, pH 7.0, 10 mM MgCl2 and 20 mM glucose) were added to the cells and they were incubated at 28° C. for 2-3 h. The transformants were selected on plates with solid LB medium supplemented with spectinomycin (50 μg/ml).

Extraction of Plasmid DNA from A. tumefaciens

To confirm that the transformed cells contained the suitable construct, plasmids were extracted from a saturated culture of the strains transformed in LB medium with spectinomycin. The cells present in 1 ml of the culture were collected by centrifugation and resuspended in 100 μl of lysis buffer (50 mM glucose, 25 mM Tris-HCl pH 7.5, 10 mM EDTA, 4 mg/ml lysozyme). The suspension was vigorously stirred for 10 min and incubated at room temperature for another 30 min, after which time 150 μl of 3M NaAc (pH 4.8) were added, it was mixed and incubated on ice for 5 min. After centrifuging at 1200 rpm for 5 min, an equal volume of phenol: chloroform (1:1) was added to the supernatant, it was mixed by stirring, and after a final centrifugation the precipitate was resuspended in 50 μl of water.

Transformation of B. napus by Floral-Dip of A. tumefaciens

B. napus was transformed by means of A. tumefaciens by the plant (in vivo) infiltration method. This method offers several advantages with respect to methods requiring an in vitro culture process. The transformation of whole plants does not require regeneration, which prevents somaclonal variation, furthermore, the time required for obtaining transformed individuals is reduced.

A saturated culture of A. tumefaciens was used to inoculate 200 ml of LB medium (3:200). This culture was incubated at 28° C. for 24 h, the cells were collected by centrifugation (3000 rpm, 15 min a 4° C.) and resuspended in 400 ml of infiltration solution (5% sucrose, 0.03% Silwet L-77 detergent (v/v), 0.5× MS medium, 0.044 μM BAP (benzylaminopurine) and acetosyringone (16 mg/l).

The B. napus plants to be infiltrated were grown in pots until they developed the floral primordia. To inoculate the plants with A. tumefaciens, 400 ml of the suspension of the bacterium were placed in a beaker, where the floral primordia of the branches of each plant were introduced one by one for 2 min, subsequently covering them with a plastic bag to maintain moisture for 2-3 days. When the bacterium is contacted with the floral primordia of B. napus, this infects the tissue. During this period of contact, the bacterium transfers to the tissue the DNA-T of the Ti plasmid, which is integrated in the genetic material of its cells, being transcribed later as if it were its own gene. Once the produced siliques were dried, the seeds were collected. The following step consisted of selecting the transgenic seeds and taking them to homozygosis.

Selection of Transgenic Seeds

After sterilizing the surface of the seeds, dormancy was eliminated by stratification for 72 h at 4° C., and they were placed on plates with MS medium supplemented with kanamycin (50 μg/ml). These plates were incubated at 22° C. with a photoperiod of 16 h of light and 8 h of darkness, and a humidity of 70% until the seedlings had completely opened cotyledons (7 days). At this time the transformed seedlings can already be distinguished from the non-transformed seedlings, since latter presented a predominant purple colour due to the accumulation of anthocyanins. The apparently transformed seedlings were transplanted in dirt, where after a few days, those not resistant to kanamycin, overlooked in the preceding step, were discarded since their primary leaves emerged white or purple.

Molecular Characterisation of the Thkel1 Transgenic Plants

The plants corresponding to lines T1 were used to check by means of PCR for the presence of the gene Thkel1 in the genome of those lines. To that end, genomic DNA was used together with specific oligonucleotides of the transgene (35S-GTW-F: CTTCGCAAGACCCTTCCTCT—SEQ ID NO: 7; Thkel1-R: GGGGACCACTTTGTACAAGAAAGCTGGGTCTTACAAAAAGTCCAACCTCC—SEQ ID NO: 8; and Thkel1-F: GGGGACAAGTTTGTACAAAAAAGCAGGCTGCATGGCCGCTTCCATCATCT—SEQ ID NO: 9). Next, the confirmed plants continued on to homozygosis.

### Example 2. Visual Observation of the Mycorrhization of Thk1 Transgenic Rapeseed Plants

To demonstrate the mycorrhization process in the Thk1 transgenic rapeseed plants of the invention, the roots thereof were submerged in a 10% KOH solution for 10-13 min at 70-80° C. to increase permeability of their cell walls. Three washes were subsequently performed with distilled water to eliminate the KOH and once more with a 2% acetic acid solution (v/v). Next, the roots were taken to a solution with Sheaffer Skrip ‘Chinese ink’ (5% of ink in 2% acetic acid) for 10 min at 90° C. Lastly, the excess ink was discarded and the roots were rinsed with distilled water, where they were kept until observation. mycorrhization was viewed with the Leica M205 FA stereoscopic microscope and photographs were taken with the Leica DFC 495 photographic camera.

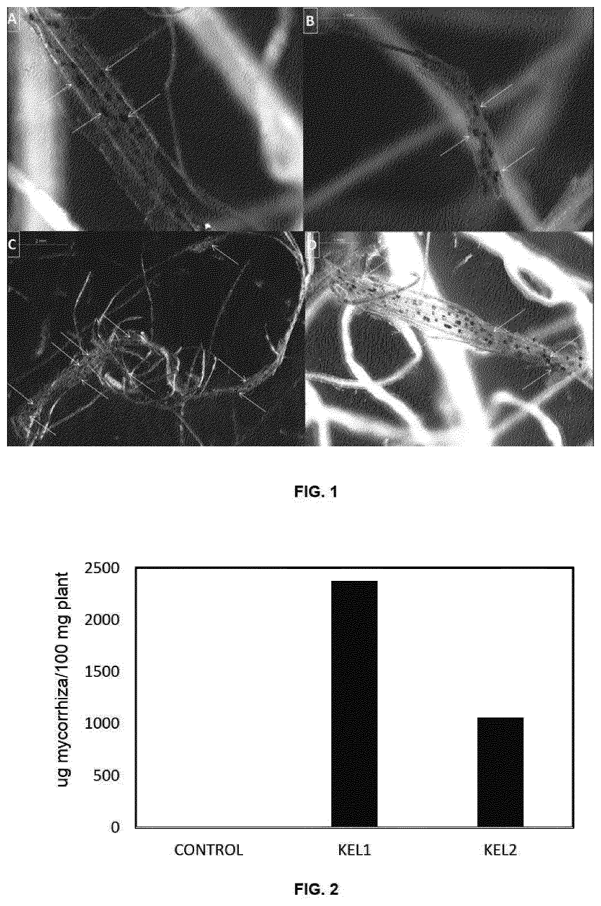

As can be observed in FIG. 1, the transgenic plants expressing the gene Thkl1 (SEQ ID NO: 1) show their roots having been subject to mycorrhization, where the vesicles formed in colonisation can be seen.

Next, mycorrhization in the transgenic rapeseed plants was quantified. By means of real-time PCR mycorrhization levels of wild-type rapeseed plants and transgenic rapeseed plants which overexpress the gene Thkel1 (SEQ ID NO: 1). As can be observed in FIG. 2, only fungal DNA was detected in the transgenic rapeseed plants.

### Example 3. Effect of the Mycorrhization on the Production of Transformed Rapeseed Plants of the Invention

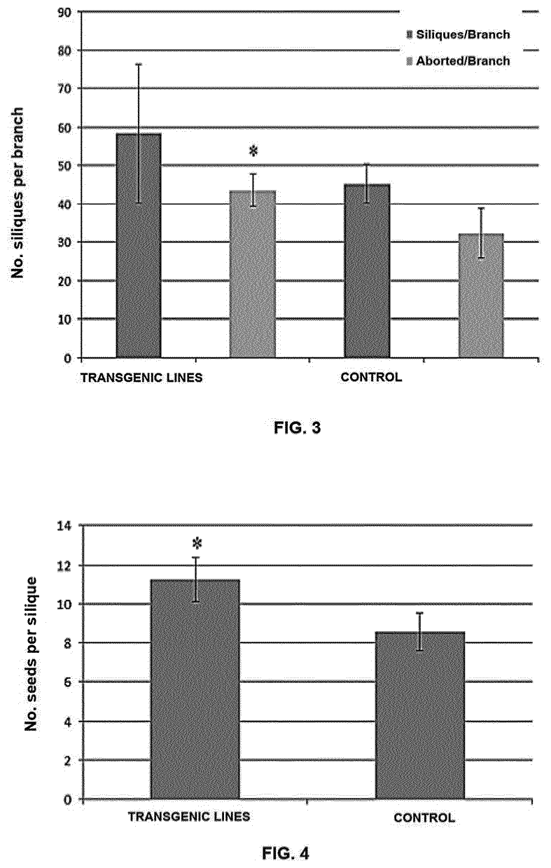

Once the first generation (T1) was obtained from a transgenic rapeseed plant which overexpresses the gene ThKel1 (SEQ ID NO: 1), the effect of mycorrhization on seed production was checked. The productive capacity of B. napus at the level of oleaginous seeds was determined by means of the collection and quantification of the total number of siliques produced by the plant. Next, each of the collected siliques was opened individually and data on the number of seeds formed in each silique was recorded, and groups of 10 seeds were weighed on a precision balance. Lastly, the weight of seeds produced by each plant was calculated. As can be seen in FIG. 3, the transgenic plants produce 30% more seeds than wild-type control plants do, and 10% more aborted seeds (flowers form completely but not fruits, siliques, or seeds) than wild-type control plants.

Moreover, it can be seen in FIG. 4 that seed production per silique in the transgenic plants subject to mycorrhization with respect to the wild-type control plants show a clear increase of 30%.

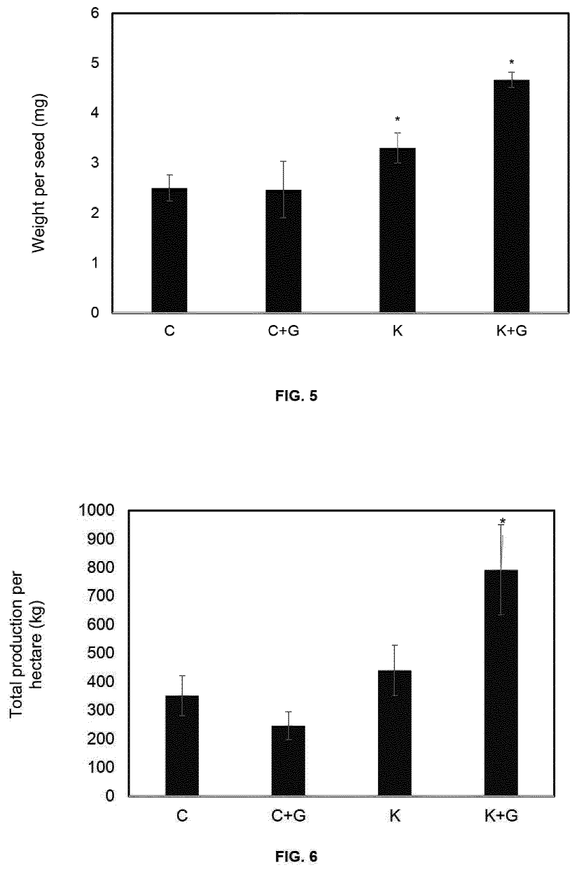

Additionally, the weight of the seeds of the transgenic plants was analysed with respect to the wild-type control plants. The results shown in FIG. 5 demonstrate that the weight of the seeds obtained from the transgenic plants is higher than the weight of the seeds from the wild-type control plants, and even more so in transgenic plants subject to mycorrhization.

With all this data, an extrapolation has been carried out to calculate the output per hectare expressed in kg (FIG. 6). This data is based on multiplying the data of the weight per seed produced per plant by the number of rapeseed plants usually existing in a hectare of this crop (about 300,000 plants, 30 per m²).

### Example 4. Effect of the Mycorrhization of Thkel1 Transgenic Rapeseed Plants in Response to Different Types of Stress

4.1 Saline Stress

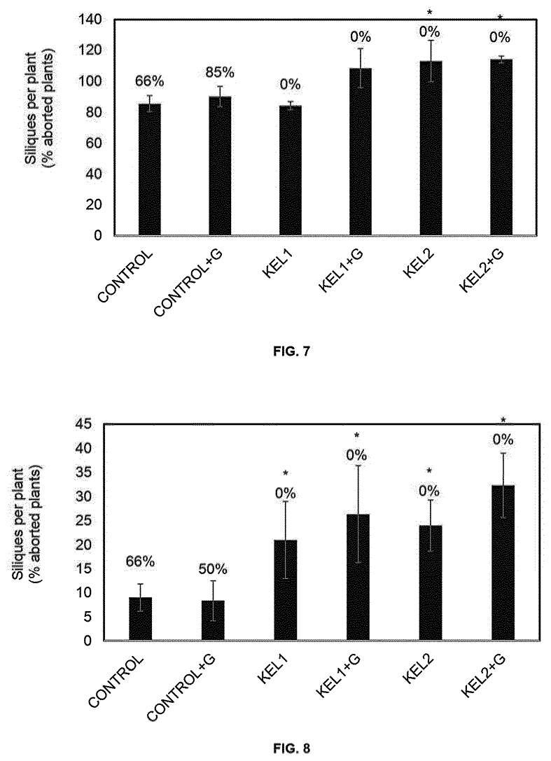

The Thkel1 transgenic rapeseed plants were grown under conditions of saline stress, generated by irrigation with 250 mM NaCl from when the sixth true leaf started to sprout in the plants. FIG. 7 shows the number of siliques per plant, including the percentage of aborted siliques, observing how all the transgenic plants produce siliques, whereas losses of around 75% were obtained in the controls. Furthermore, it shows the number of siliques produced by plant, observing how the Thkel1 transgenic plants transformed with a single copy of the gene Thkel1 (SEQ ID NO: 1) do not have a higher production than the wild-type control plants when they are grown under the same conditions of saline stress. In contrast, the presence of mycorrhizae causes a very significant increase in production, thereby clearly showing that mycorrhization does benefit the transgenic rapeseed plants of the invention subjected to saline stress.

Moreover, the Thkel2 transgenic plants that had in their genome two copies of the gene Thkel1 (SEQ ID NO: 1) have a higher production of siliques with respect to wild-type control plants, possibly due to greater β-glycosidase activity because of the presence of two copies of the gene Thkel1. However, an increase significant in production in the presence of mycorrhizae was not observed, perhaps because that greater glycosidase activity also hinders colonisation by Glomus spp.

4.2 Water Stress—Drought

Thkel1 transgenic rapeseed plants were grown under conditions of drought by means of the suspending irrigation of said plants when the sixth true leaf started to sprout. FIG. 8 shows the number of siliques per plant, including the percentage of aborted siliques, observing how the transgenic rapeseed plants of the invention produce siliques, whereas the wild-type rapeseed plants produce losses exceeding 60%. Furthermore, both the transgenic plants alone and with the transgenic plants with mycorrhizae have a significantly higher number of siliques with respect to the wild-type control plants.

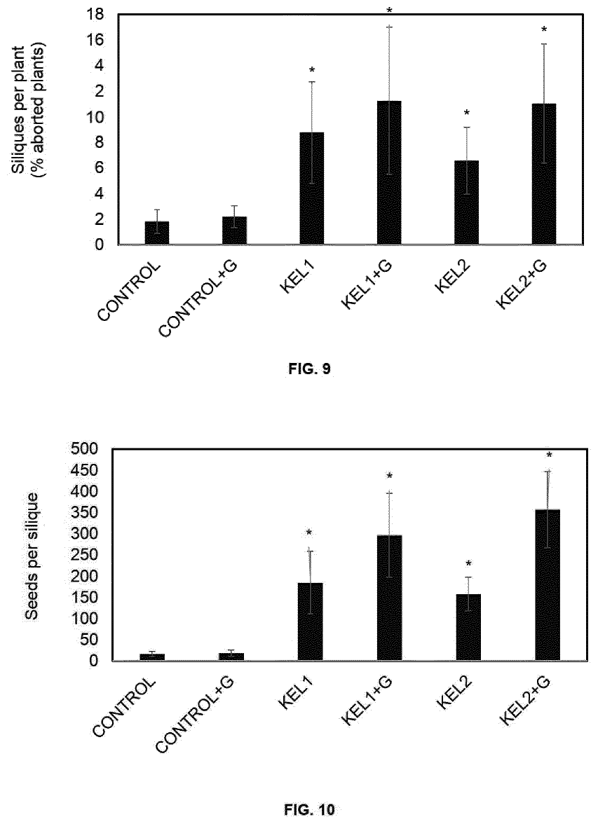

FIG. 9 shows seed production per silique of the transgenic plants of the invention with and without mycorrhizae, with respect to the wild-type control plants, with and without mycorrhizae, respectively. It can be seen in said figure how seed production of all the transgenic plants of the invention was significantly higher compared with that of the wild-type control plants, both with mycorrhizae and without mycorrhizae. Additionally, the number of seeds per plant was analysed under conditions of drought for each of the analysed plants. As can be observed in FIG. 10, seed production in all the transgenic plants was significantly higher than in the control, regardless the presence or absence of the fungus.

In FIG. 11 it could be observed how the transgenic plants of the invention, both those transformed with one and those which had two copies of the gene Thkel1 (SEQ ID NO: 1), had a higher weight compared with that of the wild-type control plants. In turn, the seeds of the transgenic plants which had two copies of the gene Thkel1 (SEQ ID NO: 1) (KEL2) in their genome showed a significantly higher weight than that of the transgenic plants with a single copy of Thkel1 (SEQ ID NO: 1) (KEL1). Moreover, upon adding mycorrhizae, no differences were observed for transgenic plants KEL1, but differences were observed in transgenic plants KEL2 with mycorrhizae with respect to the in which fungus was not added.

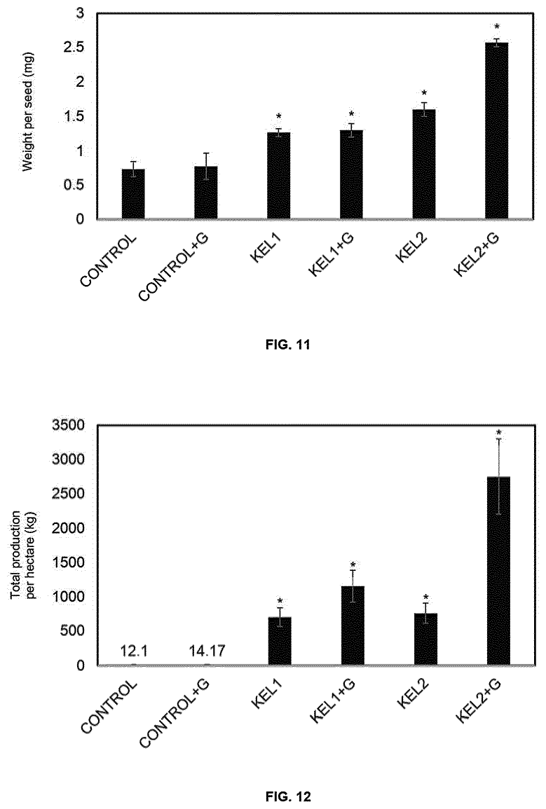

In FIG. 12, extrapolation of the output per hectare of the transgenic plants under conditions of drought and after the mycorrhization is performed. It was checked that said output was significantly higher than that of the control plants, being especially high for transgenic plants KEL2, which have in their genome two copies of the gene Thkel1 (SEQ ID NO: 1).

### Example 5. Measurement of Glucosinolates in the Thkel1 Transgenic Plants of the Invention

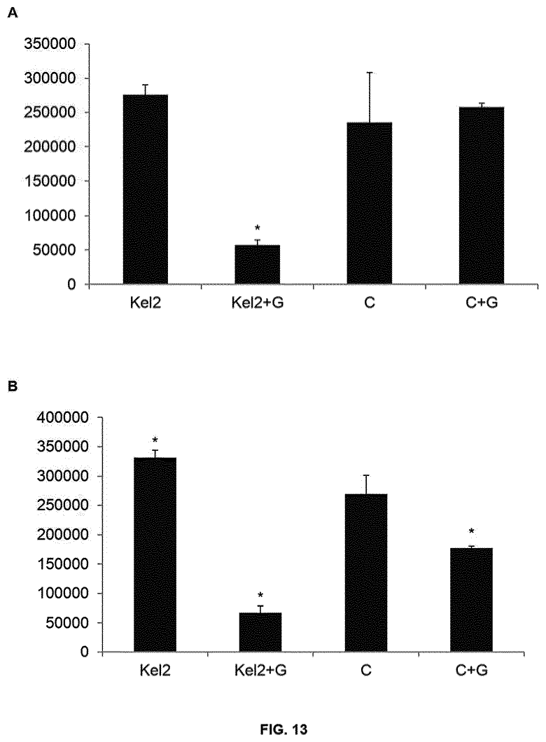

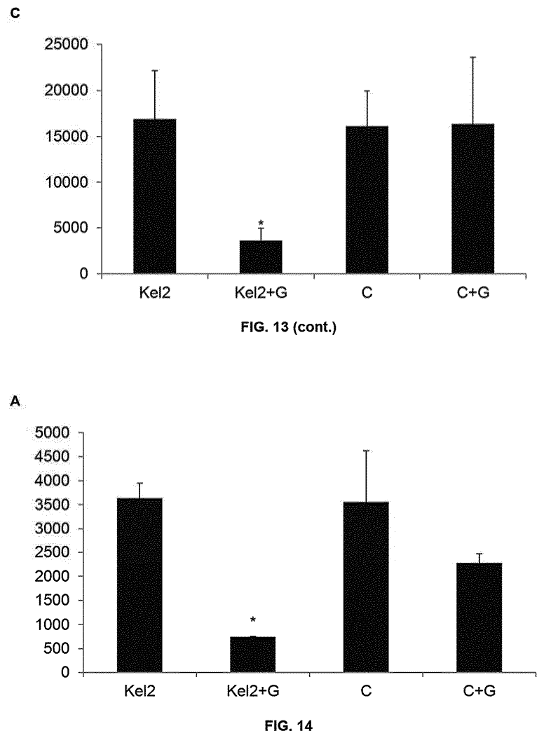

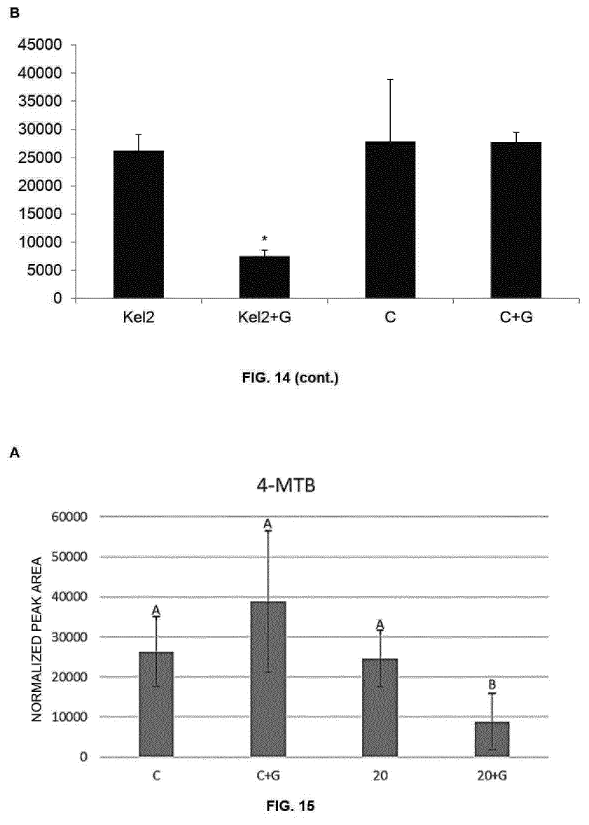

Glucosinolates were measured in the root of Thkel1 transgenic plants and wild-type plants. The results clearly show that the accumulation of indole glucosinolates, specifically 4-methoxyl-13M, 1-methoxy-13M and 4-hydroxy-13M (FIG. 13), as well as the corresponding products of their hydrolysis, 1-MeO-IGL and 4-MeO-IGL (FIG. 14), significantly decreases in the transgenic plants.

Glucosinolate hydrolysis products, 1-MeO-IGL and 4-MeO-IGL, have been related with the inability of various Cruciferae species to be subject to mycorrhization, indicating the possibility that both compounds are responsible for that impediment. Tong and others (Tong et al., Environmental and Experimental Botany. 2015; 109: 288-295), determined how, in a simultaneous broccoli (Brassica oleracea), sesame and mycorrhizal fungi crop, while the latter was being subjected to mycorrhization, there was both a root and a systemic increase in 1-MeO-IGL and 4-MeO-IGL as a defensive response of the broccoli to mycorrhization, which impeded interaction. Vierheilig and others (Vierheilig et al. New Phytologist. 2000; 146(2): 343-352) qualitatively and quantitatively measured the glucosinolate profiles of different plants belonging to the Brassicales order with and without mycorrhization capacity (in this case several Cruciferae) and observed how the difference between species of Brassicales such as Carica papaya and Tropaelum majus, with mycorrhizal capacity, and Cruciferae without mycorrhization capacity such as B. napus, B. nigra, Sinapis alba or Nastortium officinale, was the presence of these compounds.

### Example 6. Analysis of the Oleaginous Quality of the Seeds Obtained from the Thkel1 Transgenic Plants of the Invention

To determine the quality of the seeds obtained from the transgenic rapeseed plants of the invention compared with that of the seeds obtained from the wild-type rapeseed plants, compounds such as glucosinolates in the roots or fatty acids of nutritional interest present in the seeds were analysed.

For each root sample replica, 500 μl of 70% methanol supplemented with biochanin A at 1 mg/l (internal standard, IS) were added to 5 mg of tissue lyophilized plant dust. After 10 min of ultrasounds, the samples were centrifuged at 9450 g for 10 min at 4° C. Before UPLC-QTOF-MS (Ultra-High Performance Liquid Chromatography-Quadrupole Time-of-Flight Mass Spectrometry) analysis, the supernatants were filtered through PluriTetraFluoroEthylene (PTFE) syringe filters of 0.2 μm (Whatman International Inc., Kent, United Kingdom). The analyses were carried out following the polar metabolite methodology described below.

The samples of seeds were weighed (about 50 mg) and ground using a ball mile and glass beads. The resulting oily paste was mixed with 300 μl of pure methanol (liquid chromatography with mass spectrometer or LC/MS grade) complemented with biochanin A at 1 mg/l. Extraction was carried out essentially as in the root samples, but the methanol extracts were combined with 400 μl of ultrapure water and 200 μl of chloroform. The upper layer of water was recovered for polar metabolite analysis, whereas the lower organic layer was dried under vacuum and subsequently reconstituted in pure n-butanol (LC/MS grade) for non-polar metabolite analysis.

Chromatographic separations were performed in an Acquity SDS system (Waters Corp. Ltd., Milford, Mass.) interconnected to a QTOF Premier of Micromass Ltd. through an ESI source. For all separations, a Luna Omega 1.6u Polar C18 column 100 mm×2.1 mm i.d., 1.6 μm was used (Phenomenex, Torrance, U.S.A.). For data acquisition, the column was kept at 40° C. and the samples at 12° C. to slow degradation.

To analyse polar metabolites, the samples were injected into the UPLC system in aliquots of 10 μl using the partial loop filling option. The gradient elution program used was 0-2 min, 95% isocratic A [water with 0.1% formic acid (v/v)] and 5% B [acetonitrile with 0.1% formic acid (v/v)]; 2-17 min, gradient 5-95% B; 17-20 min, return to conditions initial; 20-25 min, re-equilibrium period. For acquisition, the flow rate was constant at 300 μl/min.

To analyse non-polar metabolites, the samples were injected into the UPLC system in aliquots of 5 μl using the partial loop filling option. The gradient elution program used was 0 min, 100% A [water: acetonitrile acid, 15:85 (v/v) with 0.01% formic acid and 0.5 mM CH₃COONH₄] and 0% B [n-butanol with 0.01% formic acid and 0.5 mM CH₃COONH₄]; 0-3 min, 0-10% B; 3-6 min, gradient 10-55% B; 6-9 min, gradient 55-60% B; 9-11 min, gradient 60-70% B; 11-13 return to conditions initial; and re-equilibrium. For acquisition, the flow rate was constant at 300 μl/min.

For mass spectrometry, the samples were analysed in both negative and positive ionisation modes. Two functions were established in the instrument: in function 1, data was acquired in the 50 to 1000 Da profile mode using an exploration time of 0.2 s and a collision energy of 2 eV; in function 2, The exploration range was the same, but a collision ramp was established between 4 and 65 eV. For all measurements, the capillary electrospray voltage was adjusted to 4 kV, and the cone voltage was adjusted to 25 V. The temperature of the source was kept at 120° C., and the temperature of the desolvation gas was adjusted to 350° ° C. Argon was used as the collision gas and nitrogen was used as the nebulizer, as well as desolvation gas at 60 and 800 I/h, respectively. Exact mass measurements were provided by controlling the lock mass leucine-enkephalin reference compounds.

The data was processed using Masslynx v.4.1. Raw. The data files were converted to netCDF format using the data bridge of the Masslynx application and were processed using the xcms package. Chromatographic detection of the peaks was performed using the MatchFilter 9 algorithm, applying the following configurations of parameters: snr=3, fwhm=15 s, step=0.01 D, mzdiff=0.1 D, and profmethod=bin. The retention time correction was achieved in three iterations by applying the parameters minfrac=1, bw=30 s, mzwid=0.05 D, span=1, and missing=extra=1 for the first iteration; minfrac=1, bw=10 s, mzwid=0.05 D, span=0.6, and missing=extra=0 for the second iteration; and minfrac=1, bw=5 s, mzwid=0.05 D, span=0.5, and missing=extra=0 for the third iteration. After the final maximum grouping (minfrac=1, bw=5 s) and the filling in of the missing characteristics using the fillPeaks routine in the xcms package, a data matrix consisting of the characteristic×sample was obtained.

As can be seen in FIG. 15, the mycorrhization of the transgenic plants reduces the content of all the analysed aliphatic glucosinolates in the oil of their seeds, with respect to the wild-type control plants, subject to mycorrhization or not. Moreover, it can be observed how the presence of the mycorrhizal fungus in the roots of wild-type rapeseed plants induces a significant increase in the content of the indole glucosinolate 4-MeO-13M, whereas the simple presence of the gene Thkel1 in the transgenic rapeseed plants significantly reduces this content.

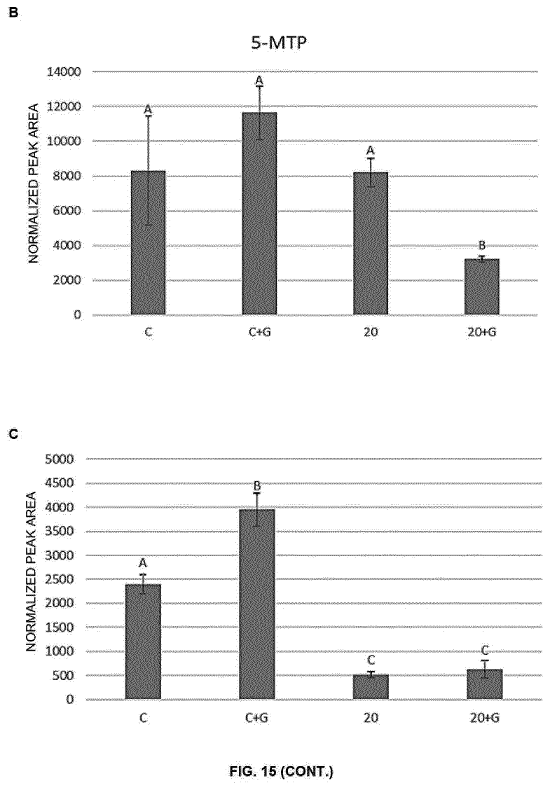

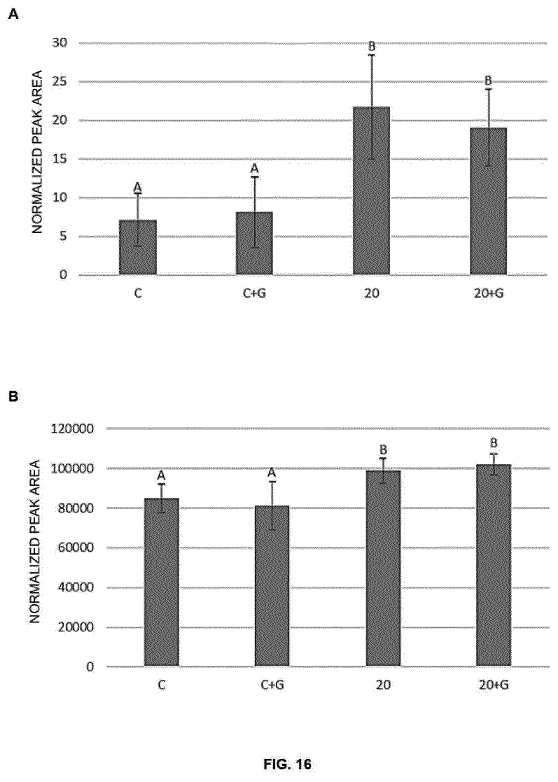

With respect to the oleic profile of the oil obtained from the seeds (FIG. 16), a significant increase in the content of octadecatrienoic acid and glycerophosphocholine in the transgenic plants of the invention with respect to the wild-type control plants can be observed. These fatty acids are of a higher quality than those present in the wild-type seeds.

1. A transgenic plant, a reproductive or propagating material, or a cultured plant cell comprising at least one copy of SEQ ID NO: 1.

2. The transgenic plant, the reproductive or propagating material, or the cultured plant cell according to claim 1, characterised in that belongs to the Brassica sp genus.

3. The transgenic plant, the reproductive or propagating material, or the cultured plant cell according to any of claims 1 to 2, characterised in that are selected from the list consisting of: B. napus, B. oleracea, B. rapa and B. nigra.

4. The transgenic plant, the reproductive or propagating material, or the cultured plant cell according to any of claims 1 to 3, characterised in that comprises more than one copy of SEQ ID NO: 1.

5. The transgenic plant, the reproductive or propagating material, or the cultured plant cell according to any of claims 1 to 4, characterised in that has mycorrhization capacity.

6. The transgenic plant, the reproductive or propagating material, or the cultured plant cell according to any of claims 1 to 5, characterised in that has higher tolerance to abiotic stress compared to the corresponding wild-type control plant.

7. The transgenic plant, the reproductive or propagating material, or the cultured plant cell according to any of claims 1 to 6, wherein the abiotic stress is selected from water stress and saline stress.

8. The transgenic plant, the reproductive or propagating material, or the cultured plant cell according to any of claims 1 to 7, characterised in that have an increased biomass, yield and/or oil compare to the corresponding wild-type control plants.

9. The transgenic plant, the reproductive or propagating material, or the cultured plant cell according to any of claims 1 to 8, characterised in that has a decreased concentration of glucosinolates compared to the corresponding wild-type control plants.

10. A cell, silique, seed, progeny or part of the plant according to any of claims 1 to 9, characterised in that comprises SEQ ID NO: 1.

11. The cell, silique, seed, progeny or part of the plant according to claim 10, wherein the parts of the plant are selected from the list consisting of: a leaf, a stem, a flower, an ovary, a fruit or a callus.

12. A use of SEQ ID NO: 1 to induce mycorrhization in plants from Brassica sp. genus.

13. The use according to claim 12, wherein the plants are selected from the list consisting of: B. napus, B. oleracea, B. rapa and B. nigra.

14. The use of SEQ ID NO: 1 for the production of transgenic plants resistant to abiotic stress, and having an increase in biomass and yield compared to the corresponding wild-type control plants.

15. A use of the transgenic plants, the reproductive or propagating material, or the cultured plant cell according to any of claims 1 to 9, and/or of the cells, siliques, seeds, progeny or part of the plant according to claims 10 to 11, for producing foods, feeds, and/or industrial products.

16. The use according to claim 15, wherein the food or feed is selected from the list consisting of: oil, semolina, grain, starch, flour or protein, and the industrial product is selected from the list consisting of: biofuel, fibre, industrial chemicals, a pharmaceutical product or a nutraceutical.

17. A method for increasing the biomass and the yield of a wild-type plant comprising:
(a) transforming said wild-type plant with an expression vector comprising the nucleotide sequence SEQ ID NO: 1, and
(b) expressing the transformed nucleic acid molecule in said plant.

18. A method for producing a transgenic plant according to any of claims 1 to 9, characterised in that it comprises the following steps:
(a) transforming a wild-type plant with an expression vector comprising the nucleotide sequence SEQ ID NO: 1, and
(b) expressing the transformed nucleic acid molecule in said plant.

19. The method according to any of claim 17 or 18, wherein the plant belongs to the Brassica spp. genus.

20. The method according to any of claims 17 to 19, characterised in that the plant is selected from the list consisting of: B. napus, B. oleracea, B. rapa and B. nigra.

21. A production method of foods, feeds and/or industrial products comprising:
(a) obtaining the plant, reproductive or propagating material, plant cell, according to any of claims 1 to 9, or cell, silique, seed, progeny or part of the plant according to any of claims 10 to 11, and
(b) preparing the food, feed and/or industrial product from the plant or part of same of step (a).

22. The method according to claim 21, wherein the food or the feed is selected from the list consisting of: oil, semolina, grain, starch, flour or protein; or the industrial product is selected from the list consisting of: biofuel, fibre, industrial chemicals, a pharmaceutical product or a nutraceutical.

23. An oil production method comprising:
(a) obtaining the seeds of the transgenic plants of claims 1 to 9, or of claim 10,
(b) grinding the seed of step (a), and
(c) extracting the oil from the seed of step (b).


SEQUENCE LISTING (9 sequences)

SEQ ID NO: 1 - DNA, 1017 bp, Trichoderma harzianum
atggccgcttccatcatctctgcccgttggaccaagcttgtggcgacccctcgcctctgccgttcttcgcatatcttgtcggccgatgcctccaagatctttatctttggtggagaactcttgccccgacagcccgtagacaaccatgtcgatcaaattgatatcgatacagagtcgaaagccaaagtcaacacactgccagtgtcagacattgcaccttcagcacgtgttggctcagctagtgtcaccatcaatggaagcatctggctcttttccggacgtggaggtctagccatggatgcgattgaagaaaatggaagcctctggcgatatgacggcacaagctccacttggcagcaaatcccccccaagtcgacagtctatccctcgtcccgcagctaccatgcaatggcattcgatggtaggaaaactatctatcttcacgctggttgcccatcccaaggacgactctccgatctgtgggcctttgataccgagacgcttgtatggactgagcatccctcagctcctgggcctgcacgcggaggaacatcaatcgctttttgcgaaggcaaactttatcgtatgggtggttttgacgggaaatcggagcaaggaggcgagcttgacatttatgatccagcctctcaggagtggcgtactattgcattcccctctgatggcatccaaggaccagaggcgcgcagtgtagcaactcttctggcagtccgactatcaagcaatgaaagtgctttggtcaccatgtttggagagcgagatccttcgtctcttgggcatgctggagctggaaagatgttggatgacgtatgggttttcaacatcaaaacctcgacctggagcaaagtcgcattccccgagcagaatgccccagcagctcgaggatggtttgcagcagatgtcatgacgcaacaaggcggggacgcggtaattgttcaagggggccttgcagaagacaacacaagattgggggatgcatggaggttggactttttgtaa

SEQ ID NO: 2 - protein, 338 aa, Trichoderma harzianum
MAASIISARWTKLVATPRLCRSSHILSADASKIFIFGGELLPRQPVDNHVDQIDIDTESKAKVNTLPVSDIAPSARVGSASVTINGSIWLFSGRGGLAMDAIEENGSLWRYDGTSSTWQQIPPKSTVYPSSRSYHAMAFDGRKTIYLHAGCPSQGRLSDLWAFDTETLVWTEHPSAPGPARGGTSIAFCEGKLYRMGGFDGKSEQGGELDIYDPASQEWRTIAFPSDGIQGPEARSVATLLAVRLSSNESALVTMFGERDPSSLGHAGAGKMLDDVWVFNIKTSTWSKVAFPEQNAPAARGWFAADVMTQQGGDAVIVQGGLAEDNTRLGDAWRLDFL

SEQ ID NO: 3 - DNA, 31 bp, Artificial Sequence
  Primer for attB1 recombination site
ggggacaagtttgtacaaaaaagcaggctgc

SEQ ID NO: 4 - DNA, 30 bp, Artificial Sequence
  Primer for attB2 recombination site
ggggaccactttgtacaagaaagctgggtc

SEQ ID NO: 5 - DNA, 29 bp, Artificial Sequence
  L1 Forward primer
tcgcgttaacgctagcatgcatggatctc

SEQ ID NO: 6 - DNA, 24 bp, Artificial Sequence
  L2 Reverse primer
gtaacatcagagattttgagacac

SEQ ID NO: 7 - DNA, 20 bp, Artificial Sequence
  35S-GTW Forward  primer
cttcgcaagacccttcctct

SEQ ID NO: 8 - DNA, 50 bp, Artificial Sequence
  Thkel1 Reverse primer
ggggaccactttgtacaagaaagctgggtcttacaaaaagtccaacctcc

SEQ ID NO: 9 - DNA, 50 bp, Artificial Sequence
  Thkel1 Forward primer
ggggacaagtttgtacaaaaaagcaggctgcatggccgcttccatcatct